(19) United States
(12) Patent Application Publication
(10) Pub. No.: US 2016/0220120 A1
(43) Pub. Date: Aug. 4, 2016

(54) OPTICAL IMAGING APPARATUS FOR MULTI-DEPTH IMAGE

(71) Applicant: POSTECH ACADEMY-INDUSTRY FOUNDATION, Pohang-si (KR)

(72) Inventors: Chul Hong KIM, Pohang-si (KR); Dong Sung KIM, Pohang-si (KR); Man Sik JEON, Daegu-si (KR); Chang Ho LEE, Daegu-si (KR); Won Kyung KIM, Pohang-si (KR)

(21) Appl. No.: 15/013,219

(22) Filed: Feb. 2, 2016

(30) Foreign Application Priority Data
Feb. 4, 2015 (KR) 10-2015-0017381

(51) Int. Cl. A61B 5/00

(52) U.S. Cl. CPC A61B 5/0095; A61B 2090/3735

(57) ABSTRACT

Provided are an optical imaging apparatus for multi-depth image and an imaging system having the same, where an optical lens of a sample stage of an optical imaging apparatus is used as a liquid lens, so that it is possible to acquire an optical image such as a photoacoustic image, an optical coherence tomography image, or a fluorescent image while finely and speedily changing a depth, and it is possible to acquire optical images having various depths by only user's simple control.

### CROSS-REFERENCE TO RELATED PATENT APPLICATION

This application claims the benefit of Korean Patent Application No. 10-2015-0017381, filed on Feb. 4, 2015 in the Korean Intellectual Property Office, the disclosure of which is incorporated herein in its entirety by reference.

### BACKGROUND OF THE INVENTION

1. Field of the Invention

The present invention relates to an optical image technique, and more particularly, optical imaging apparatus and system for multi-depth image, an optical lens of a sample stage of an optical imaging apparatus is used as a liquid lens, so that it is possible to acquire an optical image such as a photoacoustic image, an optical coherence tomography image, or a fluorescent image while finely and speedily changing a depth, and it is possible to acquire optical images having various depths by only user's simple control.

2. Description of the Related Art

In recent years, imaging systems including a high-resolution optical imaging apparatus using optical techniques are spotlighted. The high-resolution optical imaging apparatus acquires a high-resolution image of a sample by using an optical lens. As a representative imaging system described above, there are a photoacoustic imaging apparatus, an optical coherence tomography imaging apparatus, and a fluorescent imaging apparatus.

The photoacoustic imaging apparatus is a new imaging apparatus where an optical device and an ultrasonic device are combined. In the photoacoustic imaging apparatus, light energy is irradiated on an object, and the object is thermoelastically expanded according to absorption of the light energy, so that image information is generated. In addition, a recent photoacoustic imaging apparatus may employ optical laser condensing method in order to improve the image resolution, and thus, a high-resolution photoacoustic image having a scale of several micrometers can be acquired. The photoacoustic imaging apparatus is also referred to as an optical-resolution photoacoustic imaging apparatus.

In addition, the optical coherence tomography (OCT) imaging apparatus is an apparatus which provides a high-resolution OCT image by using the coherence phenomenon of light. The optical coherence tomography imaging apparatus forms an image from information obtained from scattering in an object by using a wide-band light source, an interferometer, and an optical lens. In particular, the optical coherence tomography imaging apparatus condenses light beams through the optical lens to provide the high-resolution OTC information of a scanning region.

The fluorescent imaging apparatus is an apparatus which injects a fluorescent dye generating fluorescent light at a specific wavelength into an object and examines a position, characteristics, and the like of a specific component or element. In particular, a high-resolution fluorescent microscope provides a high-resolution fluorescent image having a scale of micrometers by using an optical lens.

The high-resolution optical imaging apparatus can generate images having different depths and resolutions according to specifications of the optical lens which condenses the laser beam on a sample. Namely, the resolution and depth of the image can be configured to be variable by the optical lens installed in a sample stage.

Therefore, in the related art, in order to acquire images having various depths, optical images are acquired by replacing optical lenses having different focal lengths, or the optical images are acquired by moving the sample stage. However, in these methods, it is difficult to finely change the depth, and a long manipulation time is taken to change the depth, so that there is a problem in that the manipulation is very cumbersome.

### SUMMARY OF THE INVENTION

The present invention is to provide an optical imaging apparatus for multi-depth image and an imaging system having the same, where an optical lens of a sample stage of an optical imaging apparatus is used as a liquid lens, so that it is possible to acquire an optical image such as a photoacoustic image, an optical coherence tomography image, or a fluorescent image while finely and speedily changing a depth, and it is possible to acquire optical images having various depths by only user's simple control.

According to an aspect of the present invention, there is provided a photoacoustic imaging apparatus for multi-depth image, including: a control device which is configured to generate a control signal for depth-change corresponding to a desired depth of a photoacoustic image; a laser which generates a laser beam; a beam splitter which receives the laser beam and divides the laser beam into a first laser beam and a second laser beam; a photodetector which receives the second laser beam and generates a synchronization signal corresponding to the second laser beam to output the synchronization signal to the control device; a liquid lens device which is configured to receive and condense the first laser beam through a liquid lens and irradiate the first laser beam on the object, and which is configured to change a depth of focus of the liquid lens according to the control signal for depth-change supplied from the control device; and an ultrasonic transducer which detects a photoacoustic signal to generate electrical signal corresponding to the detected photoacoustic signal and outputs the electrical signal to the control device if the object absorbing the first laser beam generates the photoacoustic signal, wherein the control device generates and outputs a photoacoustic image by using the electrical signal provided from the ultrasonic transducer and the depth of the photoacoustic image is determined by the control signal for depth-change.

According to another aspect of the present invention, there is provided an optical coherence tomography imaging apparatus for multi-depth image, including: a control device which is configured to generate a control signal for depth-change corresponding to a desired depth of an optical coherence tomography OCT image; a laser which generates a laser beam; a liquid lens device which is configured to receive and condense a first portion of the laser beam through a liquid lens unit, irradiate the first portion of the laser beam on an object, receive and return a measurement beam obtained by deforming the first portion of the laser beam by the object, and which is configured to change a depth of focus of the liquid lens device according to the control signal for depth-change from the control device; a reference stage which receives a second portion of the laser beam, and generates and returns a reference beam; an optical fiber splitter which receives the laser beam generated by the laser to provide the first portion of the laser beam to the liquid lens device and to provide the second portion of the laser beam to the reference stage and combines the measurement beam returned from the liquid lens device and the reference beam returned from the reference stage to output combined beam; and a spectrometer which receives the combined beam of the measurement beam and the reference beam from the optical fiber splitter to acquire a coherence signal and provide the coherence signal to the control device, wherein the control device generates and outputs an OCT image by using the coherence signal provided from the spectrometer and the depth of the OCT image is determined by the control signal for depth-change.

According to still another aspect of the present invention, there is provided a fluorescent imaging apparatus for multi-depth image, including: a control device which is configured to generate a control signal for depth-change according to a desired depth of a fluorescent image; a laser which generates a laser beam; a liquid lens device which is configured to receive and condense the laser beam through a liquid lens unit, irradiate the laser beam on an object, and which is configured to change a depth of focus of the liquid lens unit according to the control signal for depth-change provided from the control device; a contrast agent which contains a fluorescent dye generating a fluorescent signal having a specific wavelength range; a filter which passes the fluorescent signal generated corresponding to irradiation of the laser beam on the object where the contrast agent is located and blocks the remaining signal; and an imaging device which images the fluorescent signal output through the filter to provide a fluorescent image information to the control device, wherein the control device outputs a fluorescent image by using the fluorescent image information provided from the imaging device and the depth of the fluorescent image is determined by the control signal for depth-change.

According to the present invention, an optical lens of a sample stage of an optical imaging apparatus is used as a liquid lens, so that it is possible to obtain the effects that it is possible to acquire an optical image such as a photoacoustic image, an optical coherence tomography image, or a fluorescent image while finely and speedily changing a depth, and it is possible to acquire optical images having various depths by only user's simple control.

### BRIEF DESCRIPTION OF THE DRAWINGS

The patent or application file contains at least one drawing executed in color. Copies of this patent or patent application publication with color drawing (s) will be provided by the Office upon request and payment of the necessary fee.

The above and other features and advantages of the present invention will become more apparent by describing in detail exemplary embodiments thereof with reference to the attached drawings in which:

FIGS. 1A to 1C are configuration diagrams illustrating a photoacoustic imaging apparatus according to an exemplary embodiment of the present invention;

FIG. 2 is a configuration diagram illustrating an optical coherence tomography imaging apparatus according to an exemplary embodiment of the present invention;

FIGS. 3A to 3C are configuration diagrams illustrating a fluorescent imaging apparatus according to an exemplary embodiment of the present invention;

FIG. 4 is a configuration diagram illustrating a control device in FIG. 1A;

FIG. 5 is a configuration diagram illustrating a liquid lens device in FIG. 1B;

FIG. 6 is a diagram illustrating a structure of a first form of a liquid lens unit according to the present invention;

FIG. 7 is a diagram illustrating an electrohydrodynamic flow in a first liquid according to operations of a control electrode in FIG. 6;

FIGS. 8A and 8B are diagrams illustrating an example of driving of the first form of the liquid lens unit in FIG. 6;

FIG. 9 is a diagram illustrating a structure of a second form of the liquid lens unit according to the present invention; and

FIGS. 10A and 10B are diagrams illustrating a structure of a third form of the liquid lens unit according to the present invention.

### DETAILED DESCRIPTION OF THE INVENTION

In the present invention, an optical lens of a sample stage of an optical imaging apparatus is used as a liquid lens, so that it is possible to acquire an optical image such as a photoacoustic image, an optical coherence tomography image, or a fluorescent image while finely and speedily changing a depth, and it is possible to acquire optical images having various depths by only user's simple control.

In particular, in the present invention, since the depth of the liquid lens is variable according to a providing time, voltage, frequency, of a control signal provided to a liquid lens unit, depth changing into user's desired depth can be speedily and accurately performed, and a degree of the depth changing can be very finely controlled. Therefore, in the diagnosis based on an optical image such as a photoacoustic image, an optical coherence tomography image, or a fluorescent image, work efficiency can be improved, and the accuracy can be improved.

<Configuration of Photoacoustic Imaging System for Multi-Depth Image>

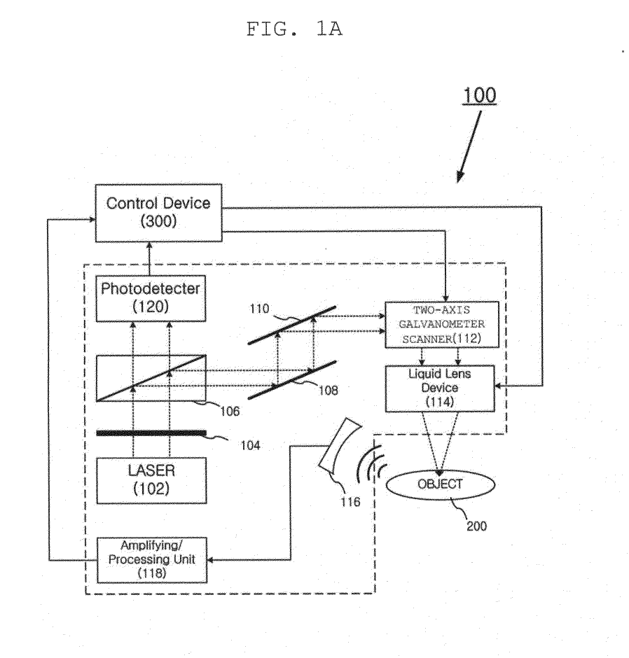

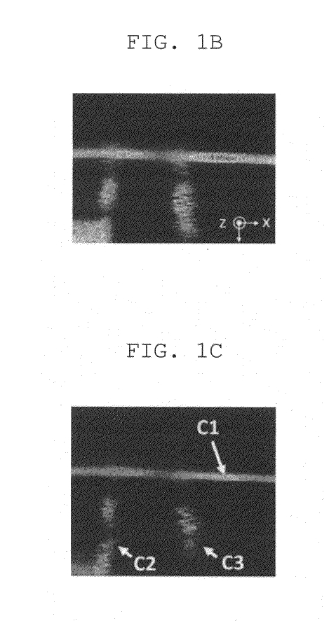

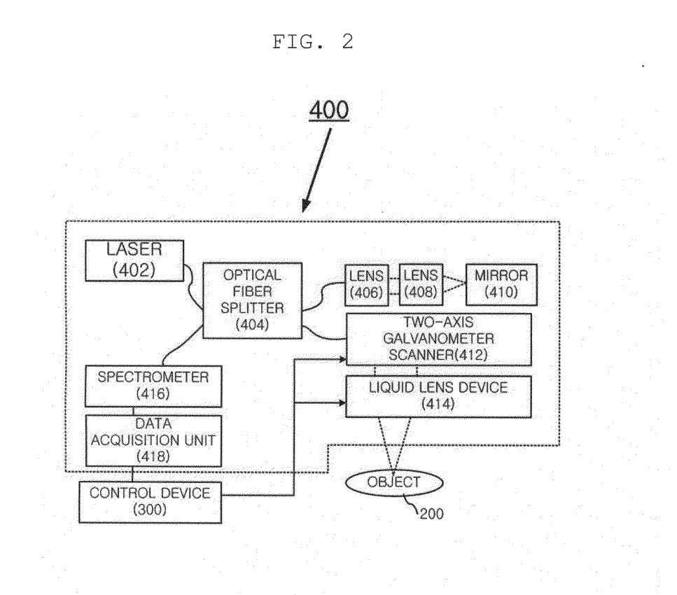

FIGS. 1A to 1C illustrate a configuration of a photoacoustic imaging apparatus for multi-depth image according to an exemplary embodiment of the present invention and photoacoustic images.

Referring to FIG. 1A, the photoacoustic imaging apparatus 100 for multi-depth image according to the present invention is configured to include a laser 102, an optical filter 104, a beam splitter 106, first and second mirrors 108 and 110, two-axis galvanometer scanner 112, liquid lens device 114, ultrasonic transducer 116, an amplifying/processing unit 118, a photodetector 120, and a control device 300.

The laser 102 generates a laser beam having a predetermined wavelength and provides the laser beam to the optical filter 104.

The optical filter 104 converts energy of the laser beam into a laser beam having energy necessary for generating of a photoacoustic image and provides the laser beam to the beam splitter 106.

The beam splitter 106 divides the laser beam into a first laser beam and a second laser beam. The first laser beam is provided to the first and second mirrors 108 and 110 in order to acquire the photoacoustic image, and the second laser beam is provided to the photodetector 120 in order to generate a synchronization signal for acquisition of a 2-D image.

The first and second mirrors 108 and 110 receive the first laser beam and change a transmission path of the first laser beam to provide the first laser beam to the two-axis galvanometer scanner 112. In this manner, the changing of the transmission path of the first laser beam by the first and second mirrors 108 and 110 is performed to acquire an optimal position in terms of a space.

The two-axis galvanometer scanner 112 two-dimensionally scans the first laser beam transmitted through the first and second mirrors 108 and 110 to acquire a 3-D photoacoustic image and provides the first laser beam to a predetermined region of an object 200 through the liquid lens device 114. Namely, the two-axis galvanometer scanner 112 is configured to include an X-axis galvanometer scanner and a Y-axis galvanometer scanner. The X-axis galvanometer scanner is driven so that the transmission path of the first laser beam is changed by a predetermined distance or the number of pieces of data of a unit pixel in order to sequentially irradiate the first laser beam along an X-axis section according to a driving signal for the scanner in synchronization with the synchronization signal. The Y-axis galvanometer scanner is driven so that the transmission path of the first laser beam is changed by the predetermined command distance or the number of pieces of data of the unit pixel every time when the driving of the X-axis galvanometer scanner is completed so that the X-axis scan along a Y-axis section according to the driving signal for the scanner in synchronization with the synchronization signal can be sequentially performed. The two-axis galvanometer scanner 112 is used for a photoacoustic imaging apparatus acquiring depth-direction image information to enable the generation of 3-D photoacoustic image information.

The liquid lens device 114 changes a depth of focus of the liquid lens unit according to a control signal for depth-change according to a user's request transmitted by the control device 300 and condenses the laser beam so as to correspond to the user's desired depth to irradiate the laser beam on the object 200. The object 200 may be a biological tissue, etc.

The object 200 absorbs the condensed laser beam and thermoelastically expands to generate a photoacoustic signal (photoacoustic wave).

The photoacoustic signal is sensed by the ultrasonic transducer 116, and the ultrasonic transducer 116 generates an electrical signal converting the photoacoustic signal and provides the electrical signal to the amplifying/processing unit 118.

The amplifying/processing unit 118 amplifies and processes the electrical signal provided from the ultrasonic transducer 116 and provides the electrical signal to the control device 300.

The photodetector 120 receives the second laser beam and generates and outputs a synchronization signal corresponding to the second laser beam.

The control device 300 as a computer or the like receives various commands or requests from the user and performs the corresponding processes. In particular, according to the exemplary embodiment, the control device receives, as input signals, a photoacoustic detection signal provided by the amplifying/processing unit 118 and a synchronization signal provided by the photodetector 120 to generate 3-D photoacoustic image information and guides the user by displaying the generated 3-D photoacoustic image information. The photoacoustic detection signal is the electrical signal converting the photoacoustic signal which is amplified and is processed.

In particular, since the control device 300 provides the driving signal for the scanner being in synchronization with the synchronization signal to the two-axis galvanometer scanner 112 and the two-axis galvanometer scanner 112 scans the first laser beam in the X-axis/Y-axis scan manner, the photoacoustic detection signal is also generated in synchronization with the synchronization signal. Therefore, the control device 300 generates 3-D photoacoustic image information by combining the photoacoustic detection signal with the synchronization signal in the X-axis and Y-axis directions.

In addition, the control device 300 provides the control signal for depth-change corresponding to the user's request to the liquid lens device 114.

FIGS. 1B and 1C are diagrams for explaining the performance of the photoacoustic imaging apparatus 100 for multi-depth image. FIG. 1B illustrates a photoacoustic image of samples before the depth of focus of the liquid lens device 114 is changed, and FIG. 1C illustrates a photoacoustic image of the samples after the depth of focus of the liquid lens device 114 is changed. In this case, a control signal for depth-change is set to have a voltage of 1.2 kVrms and a frequency of 10 Hz, and the samples are carbon fibers (C1, C2, and C3) having a diameter of 6 μm.

Although, in the photoacoustic imaging apparatus for multi-depth image according to the exemplary embodiment of the present invention, the laser beam is exemplified as a light source, it is obvious to the ordinarily skilled in the related art that an intensity modulated CW laser may be used.

<Configuration of Optical Coherence Tomography OCT Imaging Apparatus for Multi-Depth Image>

FIG. 2 illustrates a configuration of an optical coherence tomography imaging apparatus for multi-depth image according to the exemplary embodiment of the present invention and an optical coherence tomography OCT image.

Referring to FIG. 2, the optical coherence tomography imaging apparatus 400 of multi-depth image is configured to include a laser 402, an optical fiber splitter 404, lenses 406 and 408 and a mirror 410 of a reference stage, a spectrometer 416, an two-axis galvanometer scanner 412, a liquid lens device 414 of a sample stage, and a control device 300.

The laser 402 generates a laser beam having a wide band wavelength and provides the laser beam to the optical fiber splitter 404.

The optical fiber splitter 404 divides the laser beam into a third laser beam and a fourth laser beam. The third laser beam is provided to the reference stage. In the reference stage, the third laser beam is irradiated on a mirror 410 through lenses 406 and 408 to be reflected, so that reference beam is generated; and the reference beam is returned through the lenses 406 and 408 to the optical fiber splitter 404. In addition, the fourth laser beam is irradiated on an object through the two-axis galvanometer scanner 412 and the liquid lens device 414 of the sample stage to be reflected, so that measurement beam is generated; and the measurement beam is returned through the liquid lens device 414 and the two-axis galvanometer scanner 412 to the optical fiber splitter 404. Herein, the two-axis galvanometer scanner 412 two-dimensionally scans an object by using the fourth laser beam in order to acquire a 3-D optical coherence tomography image and provides the fourth laser beam to a predetermined region of the object through the liquid lens device 414.

In addition, the liquid lens device 414 changes a depth of focus of the liquid lens unit according to a control signal for depth-change provided from the control device 300 and condenses the laser beam so as to correspond to the user's desired depth of the image to irradiate the laser beam on the object.

The measurement beam is generated by scattering the condensed laser beam on the object to provide to the optical fiber splitter 404. The measurement beam and the reference beam of the reference stage at the same coherence distance combine each other to generate an optical coherence tomography signal.

The spectrometer 416 acquires beams having the coherence signal in units of a wavelength and provides the beams to a data acquisition unit 418. The data acquisition unit (frame grabber) converts the coherence signal acquired in units of a wavelength into digital coherence data and provides the digital coherence data to the control device 300.

The control device 300 as a computer or the like receives various commands from the user and performs the corresponding processes. The control device receives the coherence data acquired in units of a wavelength to generate 3-D optical coherence tomography image information and guides the user by displaying the generated 3-D optical coherence tomography image information.

<Configuration of Fluorescent Imaging Apparatus of Multi-Depth Image>

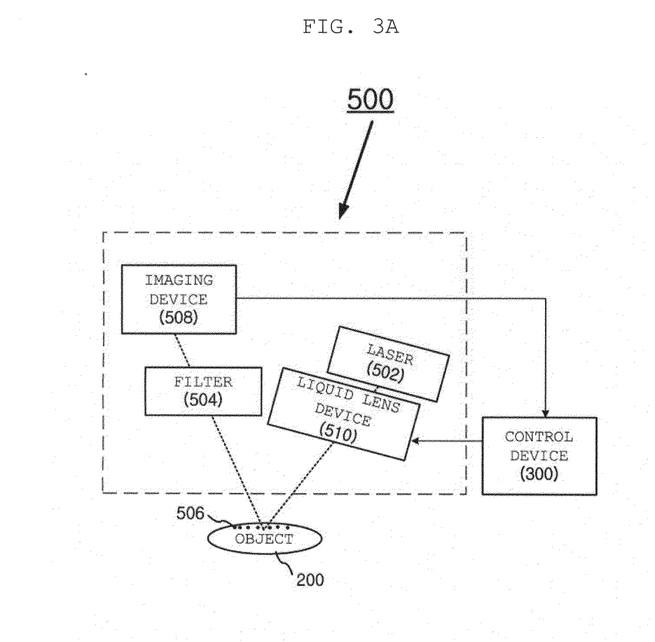

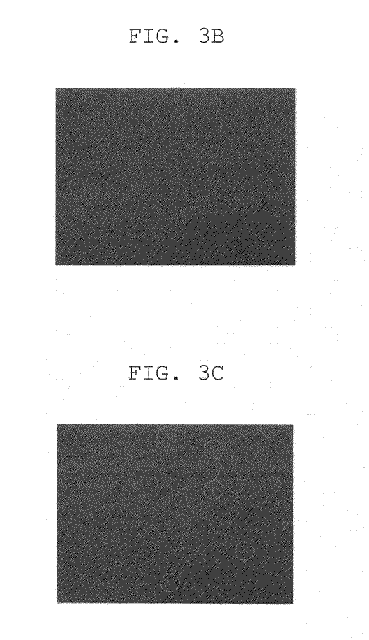

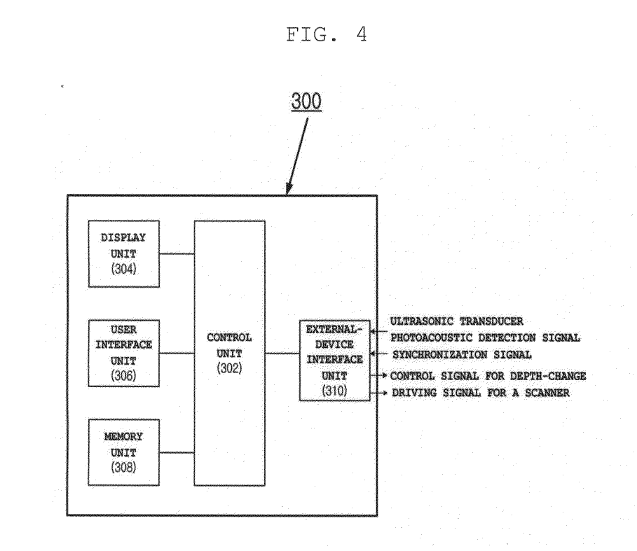

FIGS. 3A to 3C illustrate a configuration a fluorescent imaging apparatus for multi-depth image according to an exemplary embodiment of the present invention and fluorescent image information.

Referring to FIG. 3A, the fluorescent imaging apparatus 500 for multi-depth image is configured to include a laser 502, a filter 504, a contrast agent 506 containing a fluorescent dye, an imaging device 508, a liquid lens device 510, and a control device 300.

The laser 502 generates a laser beam having a short wavelength and irradiates the laser beam on the object 200 through the liquid lens device 510. In particular, the laser beam is irradiated on a region of interest where the contrast agent containing the fluorescent dye injected into the sample is located.

The laser beam irradiated on the fluorescent dye excites the fluorescent dye, and thus, the fluorescent dye generates a fluorescent signal having a visible range. The filter 504 passes the fluorescent signal and blocks the other signal to provide the fluorescent signal to the imaging device 508. The imaging device 508 images the fluorescent signal passing through the filter 504 and provides the corresponding fluorescent image information to the control device 300.

The liquid lens device 510 changes the depth of focus of the liquid lens unit according to the control signal for depth-change transmitted by the control device 300 and condenses the laser beam so as to correspond to the user's desired depth of images to irradiate the laser beam on the object.

The control device 300 generates the control signal for depth-change according to the user's request through a user interface unit (not shown) and provides the control signal for depth-change to the depth changing unit of the liquid lens device 510. The control signal for depth-change is configured with pieces of control information for changing the depth of the image into the user's desired depth. The control signal for depth-change may include information on a providing time, voltage, amplitude, frequency, and the like of the control signal which is to be provided to the control electrodes of the liquid lens unit.

FIGS. 3B and 3C are diagrams for explaining the performance of the fluorescent imaging apparatus 500 for multi-depth image according to the present invention. FIG. 3B illustrates a fluorescent image of samples before the depth of the liquid lens device 510 is changed, and FIG. 3C illustrates an optical coherence tomography image of the samples after the depth of the liquid lens device 510 is changed. In this case, a control signal for depth change is set to have a voltage of 1.2 kVrms and a frequency of 10 Hz, and the samples are fluorescent beads having a diameter of 100 μm. Herein, the fluorescent beads caused by change in depth of the liquid lens device 510 are observed in 3C.

<Configuration of Control Device>

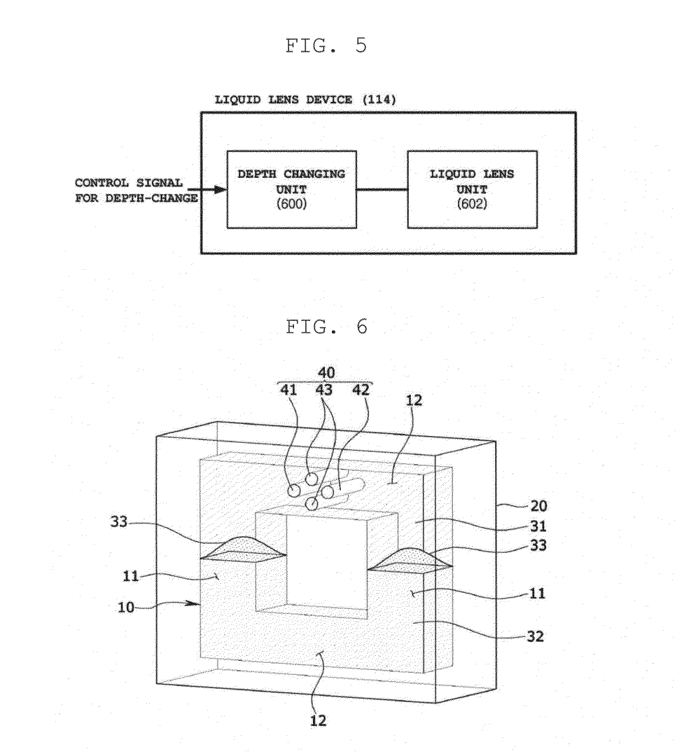

The configuration of the control device 300 will be described in detail with reference to FIG. 4.

The control device 300 is configured to include a control unit 302, a display unit 304, a user interface unit 306, a memory unit 308, and an external-device interface unit 310.

The control unit 302 receives the photoacoustic detection signal and the synchronization signal in the photoacoustic imaging apparatus 100 for multi-depth image through the external-device interface unit 310 to generate 3-D photoacoustic image information and displays the 3-D photoacoustic image information through the display unit 304.

Alternatively, the control unit receives the optical coherence tomography image information in the optical coherence tomography imaging apparatus 400 of multi-depth image and displays the optical coherence tomography image information through the display unit 304. Alternatively, the control unit receives the fluorescent image information in the fluorescent imaging apparatus 500 for multi-depth image and displays the fluorescent image information through the display unit 304.

In addition, if depth changing is requested by the user interface unit 306, the control unit 302 reads the control signal for depth-change corresponding to the requested amount of the depth changing from the memory unit 308 and provides the control signal for depth-change through the external-device interface unit 310 to the liquid lens device 114 of the photoacoustic imaging apparatus 100 for multi-depth image, the optical coherence tomography imaging apparatus 400 of multi-depth image, or the fluorescent imaging apparatus 500 for multi-depth image.

The display unit 304 outputs the image information according to the control of the control unit 302 and guides the image information to the user.

The user interface unit 306 receives various types of information and commands or requests from the user as inputs and provides the information and the commands or requests to the control unit 302.

The memory unit 308 stores various types of information including a control program of the control unit 302 and particularly stores the control signal for depth-changes corresponding to the requested amounts of the depth change. The control signal for depth-changes corresponding to the requested amounts of the depth change include applied time intervals, voltages, amplitudes, frequencies, and the like of control signals applied to control electrodes of the liquid lens unit, and the information may be acquired through experiments or the like.

The external-device interface unit 310 functions as an interface between the control unit 302 and the optical imaging apparatus 100, the optical coherence tomography imaging apparatus 400, or the fluorescent imaging apparatus 500.

<Configuration of Liquid Lens Device>

Since configurations and operations of the respective liquid lens devices 114, 414, and 510 included in the optical imaging apparatus 100, the optical coherence tomography imaging apparatus 400, and the fluorescent imaging apparatus 500 are the same, for the convenience of description, only the configuration and operations of the liquid lens device 114 will be described in detail.

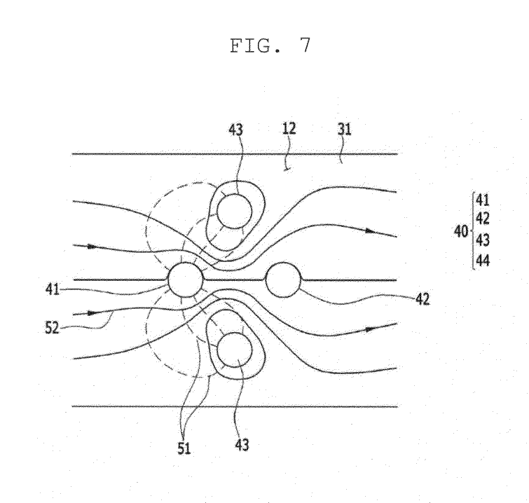

FIG. 5 illustrates the configuration of the liquid lens device 114.

The liquid lens device 114 is configured to include a depth changing unit 600 and a liquid lens unit 602.

The depth changing unit 600 receives the control signal for depth-change from the control device 300 and provides the control voltage according to the control signal for depth-change to the control electrodes installed in the liquid lens unit 602 for a time according to the control signal for depth-change to change the depth of focus of the liquid lens unit by changing the position of the lens function surface of the liquid lens unit 602 or to change the depth by changing the position and curvature of the lens function surface.

The liquid lens unit 602 as an electrohydrodynamic liquid lens changes the position of the lens function surface or changes the position and the curvature of the lens function surface according to the control voltage provided by the depth changing unit 600.

<Structure of Liquid Lens Unit>

The liquid lens unit 602 according to the present invention may have various forms, and thus, for the convenience of description, the structures of the liquid lens unit 602 according to the forms will be described.

<First Form of Liquid Lens Unit>

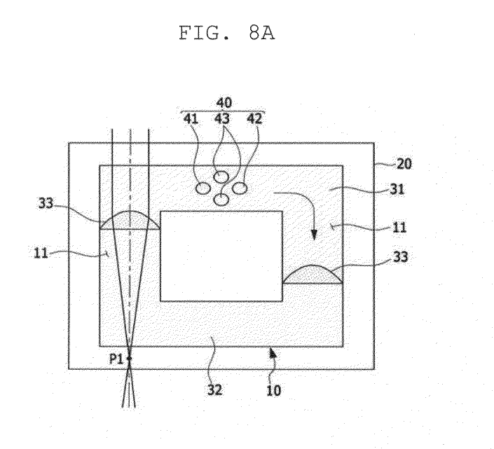

FIG. 6 illustrates a structure of a first form of the liquid lens unit according to the present invention.

The first form of the liquid lens unit is configured to include a housing 20 where a loop-shaped circulating passage 10 is formed, a first liquid 31 which is contained in a portion of the passage 10, a second liquid 32 which is contained in the remaining portion of the passage 10, and a control electrode 40 which is installed in the portion of the passage 10 where the first liquid 31 is contained. The first liquid 31 is a hydrophobic liquid, and the second liquid 32 is a hydrophilic liquid. Therefore, the first liquid and the second liquid maintain the state where the first liquid and the second liquid are not mixed but separated from each other. In addition, since the interface 33 between the first liquid 31 and the second liquid 32 functions as a lens, the interface 33 is also referred to as a lens function surface.

The housing 20 may be made of a glass, a plastic, or the like. The housing may be formed to be totally transparent, or the housing may be formed so that only a portion thereof corresponding to an optical path passing through a lens is transparent. The passage 10 inside the housing 20 is a loop-shaped circulating passage. The passage 10 may be formed in a shape of a loop with a rectangular cross section so that the passage does not communicate with the outside of the housing 20.

The first liquid 31 occupies one half of the passage 10 and the second liquid 32 occupies the remaining half of the passage 10. Wettability of the inner surface of the housing 20 to the first liquid 31 is different from wettability of the inner surface of the housing 20 to the second liquid 32. Due to a difference in wettability, curvature is formed on the interface 33 where the first liquid 31 are the second liquid 32 are in contact with each, so that the interface functions as a lens. Namely, in the case where the wettability of the inner surface of the housing 20 to the first liquid 31 is larger than the wettability to the second liquid 32, the interface 33 between the first liquid 31 and the second liquid 32 forms a curved surface convex to the first liquid 31. On the contrary, in the case where the wettability of the inner surface of the housing 20 to the first liquid 31 is smaller than the wettability to the second liquid 32, the interface 33 between the first liquid 31 and the second liquid 32 forms a curved surface convex to the second liquid 32. In particularly, as the difference between the wettability of the inner surface of the housing 20 to the first liquid 31 and the wettability to the second liquid 32 is large, the curvature of the interface 33 is formed to be large. As the first liquid 31, an insulating oil having a very low electric conductivity may be used; and as the second liquid 32, a liquid which does not mix with the first liquid 31 to form the interface 33 may be used.

The passage 10 of the housing 20 is configured to include a pair of first passages 11 where the interfaces between the first liquid 31 and the second liquid 32 are located and a pair of second passages 12 which connect the pair of first passages 11. The first passage 11 is formed in a shape of a straight line so that the interface 33 functioning as a lens is moved along the straight line.

The control electrode 40 is located in the second passage 12 filled with the first liquid 31 among the pair of second passages 12. The control electrode 40 includes first and second driving electrodes 41 and 42 which are located parallel to the longitudinal direction (horizontal direction in the figure) of the second passage 12 and a pair of ground electrodes 43 which are located parallel to the width direction (vertical direction in the figure) of the second passage 12 between the first and second driving electrodes 41 and 42. The first and second driving electrodes 41 and 42 and the pair of ground electrodes 43 are arranged in a quadrilateral shape.

During the driving process of the liquid lens 100, one of the first and second driving electrodes 41 and 42 is applied with a DC or AC voltage, and the other maintains a floating state. The direction of the flow of the first liquid 31 is determined according to which one of the first and second driving electrodes 41 and 42 is applied with the DC or AC voltage.

FIG. 7 is a partially enlarged diagram of the liquid lens unit 402 illustrated in FIG. 6, and the principle of the occurrence of the flow will be described with reference to FIG. 7. In the case where the first driving electrode 41 is applied with a high voltage and the second driving electrode 42 maintains a floating state, the pair of ground electrodes 43 maintain a ground state. Due to the application of the voltage to the first driving electrode 41, a non-uniform electric field is generated between the first driving electrode 41 and the pair of ground electrodes 43. The non-uniform electric field causes a gradient of the electric conductivity of the first liquid 31 according to the Onsager effect, and the gradient of the electric conductivity induces the generation of free electric charges in the first liquid 31 according to the Maxwell-Wagner polarization phenomenon. Free electric charges move under the influence of the electric force to transfer momentum of inertia to the surrounding fluid (first liquid), so that the flow of the first liquid 31 is generated. In 7, reference numeral 51 denotes an electric field distribution formed around the control electrode 40, and reference numeral 52 denotes a fluidic field distribution of the first liquid 31. Since the arrangement of the first driving electrode 41 and the pair of ground electrodes 43 is asymmetric in the left and right portions of FIG. 7, an electrohydrodynamic flow is generated in the horizontal direction (the longitudinal direction of the second passage 12).

Next, if the first driving electrode 41 is applied with a negative voltage, free electric charges having the opposite polarity are induced, and the direction of the electric field is also changed. As a result, the same electric force as that of the case where the first driving electrode 41 is applied with a positive voltage is exerted. Therefore, although the first driving electrode 41 is applied with an AC voltage, the flow continues to be generated constantly in the direction from the left to the right.

At this time, the first and second driving electrodes 41 and 42 and the pair of ground electrodes 43 are formed in a cylindrical shape, the resistance of the flow is reduced when the first liquid 31 flows.

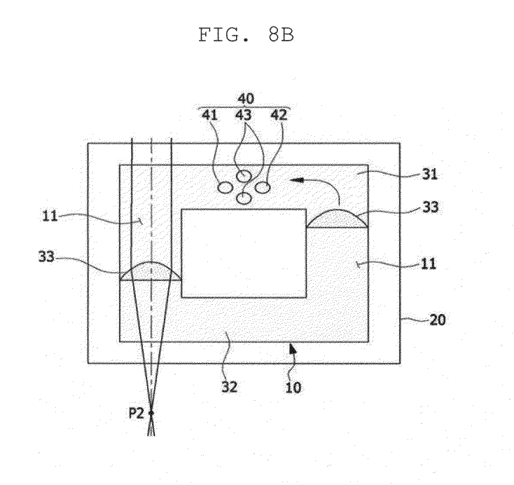

FIGS. 8A and 8B illustrates front diagrams of the liquid lens unit of FIG. 6.

In FIG. 8A, when the first driving electrode 41 is applied with a high voltage and the second driving electrode 42 maintains the floating state, the first liquid 31 flows in the direction from the first driving electrode 41 to the second driving electrode 42. Therefore, the left interface 33 of the first passage 11 moves upwards, and the right interface 33 of the first passage 11 moves downwards.

In FIG. 8B, the second driving electrode 42 is applied with a high voltage and the first driving electrode 41 maintains the floating state, the first liquid 31 flows in the direction from the second driving electrode 42 to the first driving electrode 41 (counterclockwise in the figure). Therefore, the left interface 33 of the first passage 11 moves downwards, and the right interface 33 of the first passage 11 moves upwards.

In FIGS. 8A and 8B, light incident from the outside of the housing 20 to the interface 33 is diffracted and focused while passing through the interface 33. In FIG. 8A, the light passing through the left interface 33 of the first passage 11 is focused on a point P1; and in FIG. 8B, the light passing through the left interface 33 of the first passage 11 is focused on a point P2. In FIGS. 8A and 8B, focal lengths (distance from the center of a lens to the focal point) of the lenses are the same as each other.

In this manner, by adjusting the direction of the flow of the first liquid 31, the focal point of the lens can be shifted from P1 to P2 or can be shifted from P2 to P1. Namely, the liquid lens unit can shift the focal point of the lens by using the interface 33 between the first liquid 31 and the second liquid 32 as a lens and by moving the interface 33 according to the application of the voltage to the first and second driving electrodes 41 and 42.

In addition, in this manner, after the interface 33 is moved by the flowing of the first liquid 31, if the application of the voltage to the first and second driving electrodes 41 and 42 is stopped, the flow is no longer generated, and the position of the interface 33 is also stopped. Therefore, there is no need to gradually supply energy for maintaining a desired focal point.

In particular, if the amplitude of the voltage is increased, the speed of the flow is increased; and if the frequency is increased, the speed of the flow is decreased. Therefore, by adjusting the amplitude or frequency of the voltage, the speed of the flow can be accurately controlled.

A liquid obtained by adding 0.1 wt % to 10 wt % of an additive such as an ionic surfactant, for example, sorbitane trioleate, a nonionic surfactant, for example, sodium di-2-ethylhexyl sulfossuccinat, or an oil soluble salt, for example, tetrabutylammonium tetrabutylborate to an insulating oil such as a silicon oil, dodecane, or toluene may be used as the first liquid 31.

A liquid which does not mix with the first liquid 31 to form the interface 33, for example, a liquid including a mineral oil, water, methanol, ethanol isopropyl alcohol, acetone, and the like may be used as the second liquid 32. The first liquid 31 and the second liquid 32 may have the same density.

In this case, the aforementioned lens function can be implemented irrespective of the direction of the gravity, and although the voltage is not supplied, the position and shape of the interface 33 is not changed, it is possible to reduce power consumption.

<Second Form of Liquid Lens Unit>

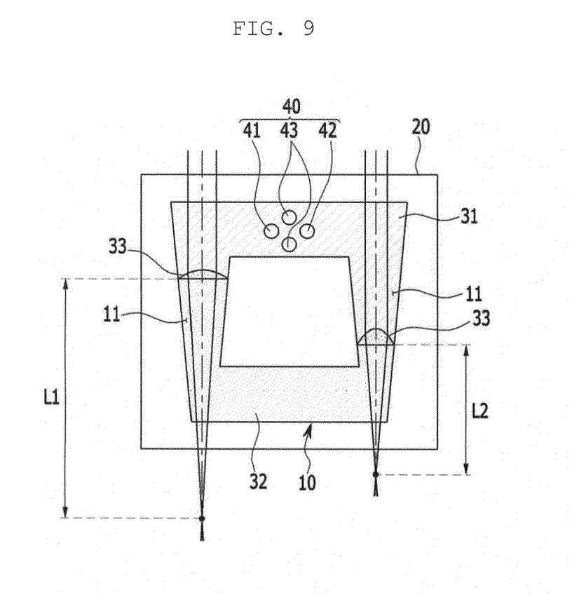

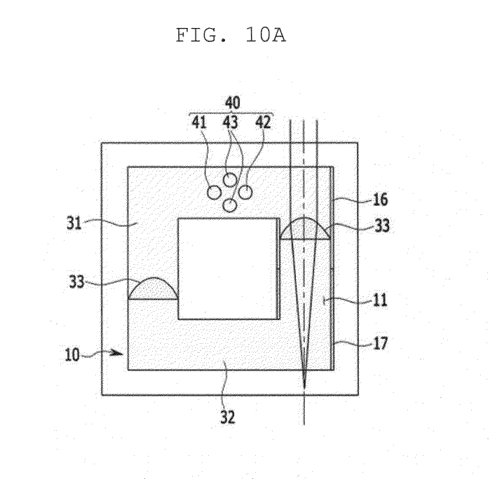

A structure of a second form of the liquid lens unit according to the present invention will be described with reference to FIG. 9.

The second form of the liquid lens unit is the same as the above-described first form except that the cross section of the first passage 11 is variable. Therefore, the same components as those of the first form are denoted by the same reference numerals, and the redundant description thereof is omitted.

The cross section of each of a pair of first passages 11 where the interfaces 33 are located is gradually changed in the longitudinal direction.

Particularly, in FIG. 9, the case where each of the first passages 11 is narrowed downwards is exemplified.

If the electrohydrodynamic flow is generated in the first liquid 31, the interface 33 between the first liquid 31 and the second liquid 32 is moved, and at this time, the curvature of the interface 33 is changed according to the position. Namely, if the interface 33 is located at the position where the cross section of the first passage 11 is large, the curvature of the interface 33 becomes small, and the focal length of the lens formed by the interface 33 becomes long. On the contrary, if the interface 33 is located at the position where the cross section of the first passage 11 is small, the curvature of the interface 33 becomes large, and the focal length of the lens formed by the interface 33 becomes short.

The two interfaces 33 formed inside the liquid lens 110 may have different curvatures according to the positions thereof. In the figure, the focal length of the left interface 33 is denoted by L1, and the focal length of the right interface 33 is denoted by L2.

In this manner, in the second form of the liquid lens unit, the focal point of the lens can be changed by moving the interface 33, and the focal length of the lens can be variously changed by changing the curvature of the interface 33. Since the shift range (distance between P1 and P2) of the focal point of the first form of the liquid lens unit is limited to the extent of the length of the first passage 11, the shift range of the focal point in the second form of the liquid lens unit is larger than that in the first form of the liquid lens unit.

In addition, in the above-described second form of the liquid lens unit, only the case where the first passage 11 is narrowed downwards is exemplified, but the first passage 11 may be formed in a shape of a venturi tube. In this case, the focal length of the lens can be more variously changed.

<Third Form of Liquid Lens Unit>

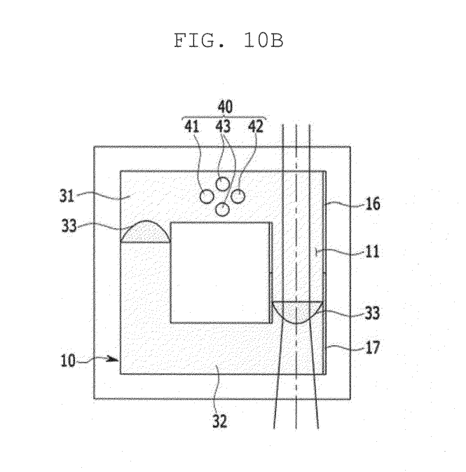

A third form of the liquid lens unit according to the present invention will be described with reference to FIGS. 10A and 10B.

The third form of the liquid lens unit is the same as the above-described first form of the liquid lens unit except that a hydrophobic surface 16 and a hydrophilic surface 17 are formed on the wall of the first passage 11. Therefore, the same components as those of the first form of the liquid lens unit are denoted by the same reference numerals, and the redundant description thereof is omitted.

In the longitudinal direction of the first passage 11, the hydrophobic surface 16 is formed on a half of the wall of the first passage 11, and the hydrophilic surface 17 is formed on the remaining half of the wall of the first passage. Namely, the hydrophobic surface 16 and the hydrophilic surface 17 are symmetric in up and down in the figure. In addition, the hydrophobic surface 16 is located closer to the control electrode 40 than the hydrophilic surface 17.

In the case where the interface between the first liquid 31 and the second liquid 32 is located in the hydrophobic surface 16 according to the flow of the first and second liquids 31 and 32, the hydrophobic surface 16 is wettable to the first liquid 31, so that the interface which is more convex upwards. Therefore, the curvature of the interface 33 is increased, so that the magnification of the lens is increased.

On the contrary, in the case where the interface between the first liquid 31 and the second liquid 32 is located in the hydrophilic surface 17, the hydrophilic surface 17 is wettable to the second liquid 32, so that the direction of the curvature of the interface 33 is reversed. Therefore, the interface 33 functioning as a focusing lens in the hydrophobic surface 16 functions as a diverging lens in the hydrophilic surface 17.

The liquid lens unit according to the present invention has features of using an insulating oil as an operating liquid (first liquid) and using an electrohydrodynamic flow. In particular, since the insulating oil is free from the problem of evaporation and has a very low electric conductivity in comparison with an aqueous solution, a flowing current is low, and power consumption is also low. In addition, since the insulating oil has a very low freezing point and a very high boiling point in comparison with an aqueous solution, the insulating oil can stably operate even in an extreme environment where external temperature is very high or low. In addition, a range of selection of the operating liquid is wide according to the density, the refractive index, and the like.

In addition, since the electrohydrodynamic flow is used, there is no need for parts such as motors or membranes. As a result, the entire structure of the liquid lens can be simplified, and manufacturing thereof can be simplified.

It is obvious to the skilled in the related art that the optical imaging apparatus according to the present invention can be applied to microscopes, endoscopes, surgical microscopes, laparoscopic devices, ophthalmometers, auriscopes, and the like.

What is claimed is:

1. A photoacoustic imaging apparatus for multi-depth image to an object to be inspected, comprising:
a control device which is configured to generate a control signal for depth-change corresponding to a desired depth of a photoacoustic image;
a laser which generates a laser beam;
a beam splitter which receives the laser beam and divides the laser beam into a first laser beam and a second laser beam;
a photodetector which receives the second laser beam and generates a synchronization signal corresponding to the second laser beam to output the synchronization signal to the control device;
a liquid lens device which is configured to receive and condense the first laser beam through a liquid lens and irradiate the first laser beam on the object, and which is configured to change a depth of focus of the liquid lens according to the control signal for depth-change supplied from the control device; and
an ultrasonic transducer which detects a photoacoustic signal to generate electrical signal corresponding to the detected photoacoustic signal and outputs the electrical signal to the control device if the object absorbing the first laser beam generates the photoacoustic signal,
wherein the control device generates and outputs a photoacoustic image by using the electrical signal provided from the ultrasonic transducer and the depth of the photoacoustic image is determined by the control signal for depth-change.

2. The photoacoustic imaging apparatus for multi-depth image according to claim 1, further comprising a scanner which receives the first laser beam from the beam splitter and changes a transmission path of the first laser beam to provide the first laser beam to the liquid lens device, so that the first laser beam is incident on a predetermined region of the object through the liquid lens device,
wherein the control device generates a driving signal for the scanner according to the synchronization signal provided from the photodetector and provides the driving signal for the scanner to the scanner; and
wherein the scanner is driven by the driving signal for the scanner provided from the control device.

3. The photoacoustic imaging apparatus for multi-depth image according to claim 1, wherein the liquid lens device is configured to include:
a liquid lens unit including:
a housing where a circulating passage is formed;
a first hydrophobic liquid which is contained in a portion of the passage;
a second hydrophilic liquid which is contained in the remaining portion of the passage and maintains a state that the second liquid and the first liquid are separated from each other; and
a control electrode which is installed in the portion of the passage where the first liquid is contained; and
a depth changing unit which is configured to be provided the control signal for depth-change from the control device, and provide a control voltage according to the control signal for depth-change to the control electrode,
wherein, due to a difference between wettability of the inner surface of the housing to the first liquid and wettability of the inner surface of the housing to the second liquid, a curvature is formed on an interface where the first liquid and the second liquid are in contact with each other, so that the interface functions as a lens, and during application of the control voltage through the control electrode, an electrohydrodynamic flow is generated in the first liquid, and thus, the position of the interface is changed, so that the depth of focus of the liquid lens unit is changed.

4. The photoacoustic imaging apparatus for multi-depth image according to claim 3,
wherein a cross section of a portion where the interface is moved in the passage is formed to be constant, so that the position of the interface functioning as a lens due to the flow of the first liquid is changed, or
wherein the cross section of the portion where the interface is moved in the passage is formed to be changed into a predetermined shape, so that the position and curvature of the interface functioning as a lens due to the flow of the first liquid are changed.

5. The photoacoustic imaging apparatus for multi-depth image according to claim 3,
wherein a portion of an inner surface of the portion where the interface is moved in the passage is formed with a hydrophobic surface,
wherein the remaining portion of the inner surface of the portion where the interface is moved in the passage is formed with a hydrophilic surface, and
wherein the position and curvature of the interface functioning as a lens due to the flow of the first liquid are changed.

6. The photoacoustic imaging apparatus for multi-depth image according to claim 3,
wherein the first liquid is an insulating oil, and
wherein the second liquid contains one of a mineral oil, water, methanol, ethanol, isopropyl alcohol, and acetone.

7. The photoacoustic imaging apparatus for multi-depth image according to claim 1,
wherein the control device includes a user interface unit; and
wherein when a change of depth is requested by the user interface unit, the control device generates a control signal for depth-change corresponding to a desired level of depth and provides the control signal to the liquid lens device.

8. The photoacoustic imaging apparatus for multi-depth image according to claim 1, wherein the photoacoustic imaging apparatus for multi-depth image is used for a microscope, an endoscope, a surgical microscope, a laparoscopic device, an ophthalmometer, or an auriscope.

9. An optical coherence tomography imaging apparatus for multi-depth image to an object to be inspected, comprising:
a control device which is configured to generate a control signal for depth-change corresponding to a desired depth of an optical coherence tomography OCT image;
a laser which generates a laser beam;
a liquid lens device which is configured to receive and condense a first portion of the laser beam through a liquid lens unit, irradiate the first portion of the laser beam on an object, receive and return a measurement beam obtained by deforming the first portion of the laser beam by the object, and which is configured to change a depth of focus of the liquid lens device according to the control signal for depth-change from the control device;
a reference stage which receives a second portion of the laser beam, and generates and returns a reference beam;
an optical fiber splitter which receives the laser beam generated by the laser to provide the first portion of the laser beam to the liquid lens device and to provide the second portion of the laser beam to the reference stage and combines the measurement beam returned from the liquid lens device and the reference beam returned from the reference stage to output combined beam; and
a spectrometer which receives the combined beam of the measurement beam and the reference beam from the optical fiber splitter to acquire a coherence signal and provide the coherence signal to the control device,
wherein the control device generates and outputs an OCT image by using the coherence signal provided from the spectrometer and the depth of the OCT image is determined by the control signal for depth-change.

10. The optical coherence tomography imaging apparatus for multi-depth image according to claim 9, further comprising a scanner which receives the first portion of the laser beam from the optical fiber splitter and changes a transmission path of the laser beam to provide the first portion of the laser beam to the liquid lens device, so that the first portion of the laser beam is incident on a predetermined region of the object through the liquid lens device,
wherein the control device generates a driving signal for the scanner and provides the driving signal for the scanner; and
wherein the scanner is driven by the driving signal for the scanner provided from the control device.

11. The optical coherence tomography imaging apparatus for multi-depth image according to claim 9,
wherein the control device includes a user interface unit; and
wherein when change of depth is requested by the user interface unit, the control device generates a control signal for depth-change corresponding to a desired level of depth and provides the control signal for depth-change to the liquid lens device.

12. The optical coherence tomography imaging apparatus for multi-depth image according to claim 9,
wherein the liquid lens device is configured to include:
a liquid lens unit including:
a housing where a circulating passage is formed;
a first hydrophobic liquid which is contained in a portion of the passage;
a second hydrophilic liquid which is contained in the remaining portion of the passage and maintains a state that the second liquid and the first liquid are separated from each other; and
a control electrode which is installed in the portion of the passage where the first liquid is contained; and
a depth changing unit which is configured to be provided the control signal for depth-change from the control device, and provide a control voltage according to the control signal for depth-change to the control electrode, and
wherein, due to a difference between wettability of the inner surface of the housing to the first liquid and wettability of the inner surface of the housing to the second liquid, a curvature is formed on an interface where the first liquid and the second liquid are in contact with each other, so that the interface functions as a lens, and during application of a control signal through the control electrode, an electrohydrodynamic flow is generated in the first liquid, and thus, the position of the interface is changed, so that the depth of focus of the liquid lens unit is changed.

13. The optical coherence tomography imaging apparatus for multi-depth image according to claim 12,
wherein a cross section of a portion where the interface is moved in the passage is formed to be constant, so that the position of the interface functioning as a lens due to the flow of the first liquid is changed, or
wherein the cross section of the portion where the interface is moved in the passage is formed to be changed into a predetermined shape, so that the position and curvature of the interface functioning as a lens due to the flow of the first liquid is changed.

14. The optical coherence tomography imaging apparatus for multi-depth image according to claim 12,
wherein a portion of an inner surface of the portion where the interface is moved in the passage is formed with a hydrophobic surface,
wherein the remaining portion of the inner surface of the portion where the interface is moved in the passage is formed with a hydrophilic surface, and
wherein the position and curvature of the interface functioning as a lens due to the flow of the first liquid are changed.

15. The optical coherence tomography imaging apparatus for multi-depth image according to claim 12,
wherein the first liquid is an insulating oil, and
wherein the second liquid contains one of a mineral oil, water, methanol, ethanol, isopropyl alcohol, and acetone.

16. A fluorescent imaging apparatus for multi-depth image for multi-depth image to an object to be inspected, comprising:
a control device which is configured to generate a control signal for depth-change according to a desired depth of a fluorescent image;
a laser which generates a laser beam;
a liquid lens device which is configured to receive and condense the laser beam through a liquid lens unit, irradiate the laser beam on an object, and which is configured to change a depth of focus of the liquid lens unit according to the control signal for depth-change provided from the control device;
a contrast agent which contains a fluorescent dye generating a fluorescent signal having a specific wavelength range;
a filter which passes the fluorescent signal generated corresponding to irradiation of the laser beam on the object where the contrast agent is located and blocks the remaining signal; and
an imaging device which images the fluorescent signal filtered by the filter to provide a fluorescent image information to the control device,
wherein the control device outputs a fluorescent image by using the fluorescent image information provided from the imaging device and the depth of the fluorescent image is determined by the control signal for depth-change.

17. The fluorescent imaging apparatus for multi-depth image according to claim 16,
wherein the liquid lens device is configured to include:
a liquid lens unit including:
a housing where a circulating passage is formed;
a first hydrophobic liquid which is contained in a portion of the passage;
a second hydrophilic liquid which is contained in the remaining portion of the passage and maintains a state that the second liquid and the first liquid are separated from each other; and
a control electrode which is installed in the portion of the passage where the first liquid is contained; and
a depth changing unit which is configured to be provided the control signal for depth-change from the control device, and provide a control voltage according to the control signal for depth-change to the control electrode, and
wherein, due to a difference between wettability of the inner surface of the housing to the first liquid and wettability of the inner surface of the housing to the second liquid, a curvature is formed on an interface where the first liquid and the second liquid are in contact with each other, so that the interface functions as a lens, and during application of a control signal through the control electrode, an electrohydrodynamic flow is generated in the first liquid, and thus, the position of the interface is changed, so that the depth of focus of the liquid lens unit is changed.

18. The fluorescent imaging apparatus for multi-depth image according to claim 17,
wherein a cross section of a portion where the interface is moved in the passage is formed to be constant, so that the position of the interface functioning as a lens due to the flow of the first liquid is changed, or
wherein the cross section of the portion where the interface is moved in the passage is formed to be changed into a predetermined shape, so that the position and curvature of the interface functioning as a lens due to the flow of the first liquid is changed.

19. The fluorescent imaging apparatus for multi-depth image according to claim 17,
wherein a portion of an inner surface of the portion where the interface is moved in the passage is formed with a hydrophobic surface,
wherein the remaining portion of the inner surface of the portion where the interface is moved in the passage is formed with a hydrophilic surface, and
wherein the position and curvature of the interface functioning as a lens due to the flow of the first liquid are changed.

20. The fluorescent imaging apparatus for multi-depth image according to claim 17,
wherein the first liquid is an insulating oil, and
wherein the second liquid contains one of a mineral oil, water, methanol, ethanol, isopropyl alcohol, and acetone.
